(19) United States
(12) Patent Application Publication
(10) Pub. No.: US 2026/0007801 A1
(43) Pub. Date: Jan. 8, 2026

(54) METHOD AND DEVICE FOR A METHOD FOR PREPARING A RECOMBINANT COLLAGEN HYDROGEL TISSUE ADHESIVE FOR SEAMLESS WOUND HEALING

(71) Applicant: Northwest University, Xi'an (CN)

(72) Inventors: Dan Zheng, Xi'an (CN); Rui He, Xi'an (SN); Daidi Fan, Xi'an (SN); Zewen Chang, Xi'an (CN); Ruiyao Wang, Xi'an (CN); Shuchang Wei, Xi'an (CN)

(73) Assignee: Northwest University, Xi'an, SN (CN)

(21) Appl. No.: 19/286,217

(22) Filed: Jul. 30, 2025

(30) Foreign Application Priority Data
Oct. 8, 2024 (CN) 202411388407.6

(51) Int. Cl. A61L 24/04; A61L 24/00; C08L 5/08; C08L 71/02; C08L 79/02; C08L 89/06; C12N 9/10

(52) U.S. Cl. CPC A61L 24/043; A61L 24/0031; C08L 5/08; C08L 71/02; C08L 79/02; C08L 89/06; C12N 9/1044; C12Y 203/02013

(57) ABSTRACT

The invention discloses a preparation method for a recombinant collagen hydrogel tissue adhesive for seamless wound healing. The invention uses recombinant collagen as the main component and utilizes the synergistic effects of various high-molecular polymers to achieve rapid formation of hydrogels with excellent adhesion, antibacterial properties, promotion of collagen deposition at wound sites, and controllable biodegradability. The prepared double-network hydrogel tissue adhesive can be injected into deep wounds, replacing sutures and staples in wound closure applications, with many practical applications.

### TECHNICAL FIELD

The present invention pertains to the field of biomaterials technology, specifically involving a preparation method for a recombinant collagen hydrogel tissue adhesive used for sutureless wound healing.

### BACKGROUND ART

Each year, millions of people suffer from various types of wounds, including those caused by accidental trauma and surgical incisions. Post-tissue injury, significant bleeding and wound infection are primary causes of death. Currently, wound closure devices commonly used include staples, sutures, adhesives, sealants, and mechanical devices. Among these, surgical sutures and staples play crucial roles in clinical procedures. However, their drawbacks such as time-consuming operation, poor aesthetic outcomes, and suboptimal tissue integration no longer meet the growing demand for effective wound healing. In recent years, tissue sealants and adhesives have gradually emerged as alternatives to sutures and staples for wound or incision closure and sealing. These materials offer advantages such as minimal invasiveness, lower professional requirements, shorter application times, more ideal tissue integration, and reduced tissue damage.

Commercially available adhesives currently on the market primarily fall into three categories: bio-based adhesives, synthetic organic adhesives, and composite adhesives. Naturally occurring adhesives, such as mussel foot protein adhesive and fibrin glue, exhibit excellent biocompatibility and biodegradability; however, their adhesive performance in physiological environments is weak due to their reliance on relatively weak physical interactions, typically resulting in low adhesion values ranging from 1 to 10 J/m². On the other hand, synthetic organic adhesives like cyanoacrylate glues, although exhibiting higher adhesive properties, are limited in clinical applications within tissues due to the toxicity of cyanoacrylates.

Therefore, there remains a need for superior tissue adhesives that combine reliable adhesive performance with good biocompatibility, biodegradability, hemostatic activity, and antimicrobial properties, serving as convenient options for trauma emergency care and surgical incision adhesion.

### SUMMARY

The present invention aims to address the deficiencies of existing technologies by providing a method for preparing a recombinant collagen hydrogel tissue adhesive for seamless wound healing. This method uses recombinant collagen as the main component and leverages the synergistic effects of various high-molecular-weight polymers to rapidly form a hydrogel with excellent adhesion, antibacterial properties, and the ability to promote collagen deposition at the wound site, as well as controllable biodegradability. The prepared double-network hydrogel tissue adhesive can be injected into deep wounds, replacing sutures and staples in wound closure applications, and has promising application prospects.

To solve the aforementioned technical problems, the technical solution of this invention is as follows: A method for preparing a recombinant collagen hydrogel tissue adhesive for seamless wound healing, characterized by the following steps:
Step one: At room temperature, dissolve carboxymethyl chitosan and polylysine in deionized water to obtain a mixed solution, adjust the pH value of the mixed solution to neutral, then add transglutaminase to the mixed solution to obtain Solution A;
Step two: Dissolve recombinant collagen and tetra-arm-polyethylene glycol-aldehyde in deionized water to obtain Solution B;
Step three: Mix Solution A from Step one and Solution B from Step two evenly to obtain a recombinant collagen hydrogel tissue adhesive for seamless wound healing.

The method for preparing a recombinant collagen hydrogel tissue adhesive for seamless wound healing, wherein in Step one, the mass percentage content of carboxymethyl chitosan in the mixed solution is 8.4% to 12%, and the mass percentage content of polylysine is 6.7% to 9.5%.

The method for preparing a recombinant collagen hydrogel tissue adhesive for seamless wound healing, wherein in Step one, the amount of transglutaminase is adjusted according to the mass of recombinant collagen, with no less than 0.08 g of transglutaminase per gram of recombinant collagen.

The method for preparing a recombinant collagen hydrogel tissue adhesive for seamless wound healing, wherein in Step two, the content of recombinant collagen in Solution B is adjusted based on the required time for wound healing, with a mass percentage content of recombinant collagen not less than 8%, and the mass percentage content of tetra-arm-polyethylene glycol-aldehyde is 8% to 12%.

The method for preparing a recombinant collagen hydrogel tissue adhesive for seamless wound healing, wherein the mass percentage content of recombinant collagen in Solution B is 8% to 15%.

The method for preparing a recombinant collagen hydrogel tissue adhesive for seamless wound healing, wherein in Step two, the recombinant collagen is CF-1552 type I recombinant collagen, and the molecular weight of tetra-arm-polyethylene glycol-aldehyde is 10,000.

The method for preparing a recombinant collagen hydrogel tissue adhesive for seamless wound healing, wherein in Step three, the volume ratio of Solution A to Solution Bis 1:1.

The method for preparing a recombinant collagen hydrogel tissue adhesive for seamless wound healing, wherein in Step three, a three-way valve and a syringe are used to mix Solution A and Solution B evenly.

The present invention has the following advantages compared to existing technologies:

1. The invention utilizes recombinant collagen (especially CF-1552 type I recombinant collagen from the College of Chemical Engineering, Biomedical Research Institute at Northwest University, which contains a large amount of glutamine) to form amide bonds with poly-L-lysine (EPL) through enzymatic reactions, and by forming a double network, it retains its low immunogenicity while imparting strong adhesion, antibacterial properties, self-healing ability, injectability, and controllable biodegradability to the adhesive.

2. The invention employs a combination of transglutaminase (TGase)-mediated enzymatic reaction and Schiff base reaction to quickly form a tissue adhesive with wound adaptability and certain mechanical properties via the first network, and then enhances its adhesion performance and controls its degradation rate through the second network. This dual-network structure addresses the issues of long gelation time, poor mechanical properties, lack of fatigue toughness, excessive swelling, rapid degradation rate of single-network collagen, and instability, poor adhesion, flexibility, and toughness of single-network Schiff bases.

3. For the first time, the invention uses carboxymethyl chitosan (CMCS) and poly-L-lysine (EPL) together to provide amino groups for synthesizing hydrogel adhesives. By leveraging the synergistic effect of CMCS and EPL, it solves the problem of non-adhesive hydrogels formed by CMCS and aldehyde groups, as well as the long gelation time of hydrogels formed by EPL and aldehyde groups.

4. The invention uses tetra-arm-polyethylene glycol-aldehyde as a polymer to provide a large number of aldehyde groups for the formation of the Schiff base network in hydrogels, forming reversible Schiff base bonds with amino groups to endow hydrogels with injectability and self-healing ability, while enhancing their adhesion to tissues.

5. The invention preferably uses a three-way valve and syringe to mix Solution A and Solution B, allowing rapid uniform mixing of the two solutions and achieving injectability of the tissue adhesive by connecting the syringe to the three-way valve, thereby effectively closing deep wounds.

6. The preparation method of the invention is simple, does not require any chemical cross-linkers, and produces recombinant collagen hydrogel tissue adhesives with high biological compatibility and reproducibility, capable of tightly bonding wounds with strong adhesion, robust antibacterial properties, and low immunogenicity. It can replace sutures and staples to promote the repair of deep wounds caused by surgical incisions and accidental trauma, demonstrating good application prospects.

Below, the technical solution of the invention is further detailed with reference to the drawings and examples.

### BRIEF DESCRIPTION OF THE DRAWINGS

FIG. 1 is a flowchart illustrating the process for preparing hydrogel tissue adhesives according to the present invention.

FIG. 2 shows the appearance of A solution, B solution, and their mixture at room temperature according to the present invention.

FIG. 3 presents the FTIR spectra of various networks in the hydrogel tissue adhesive prepared according to the present invention.

FIG. 4 displays the SEM images of various networks in the hydrogel tissue adhesive prepared according to the present invention.

FIG. 5 shows the adhesion performance test results of the hydrogel tissue adhesive prepared according to the present invention.

FIG. 6 presents the self-healing test results of the hydrogel tissue adhesive prepared according to the present invention.

FIG. 7 illustrates the injectability test results of the hydrogel tissue adhesive prepared according to the present invention.

FIG. 8 shows the cytotoxicity test results of the hydrogel tissue adhesive prepared according to the present invention.

FIG. 9 presents the antibacterial test results of the hydrogel tissue adhesive prepared according to the present invention.

FIG. 10 displays the wound closure and healing test results of the hydrogel tissue adhesive prepared according to the present invention.

### DETAILED DESCRIPTION OF THE EMBODIMENTS

To make the aforementioned objectives, features, and advantages of this invention more apparent and understandable, detailed descriptions of specific implementations of this invention are provided below in conjunction with examples. In the following examples, the molecular weight of carboxymethyl chitosan is 544, and the molecular weight of poly-L-lysine ranges from 2000 to 5000, both purchased from Aladdin; transglutaminase (TGase) has an enzyme activity of 1000 U/g, purchased from Solarbio; recombinant collagen is CF-1552 type I recombinant collagen, sourced from the Institute of Biomedical Engineering at the College of Chemistry and Chemical Engineering, Northwest University. CF-1552 type I recombinant collagen is derived from human mRNA reverse-transcribed into cDNA and expressed in Escherichia coli BL21. Its structure and acquisition method can be referenced in patent application number ZL01106757.8 titled ‘A Human-like Collagen and Its Production Method’ or in the literature Recombinant collagen for the repair of skin wounds and photo-aging damage, Regenerative Biomaterials, 2024, 11, rbae108. Tetra-arm PEG-aldehyde has a molecular weight of 10000, purchased from Macklin. Unless otherwise specified, all reagents used in this invention description are commercially available.

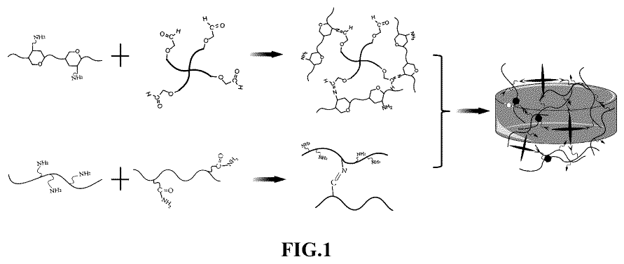

The preparation method of the recombinant collagen hydrogel tissue adhesive for sutureless wound healing according to this invention is illustrated in FIG. 1, specifically including the following steps:

Step one: At room temperature, dissolve carboxymethyl chitosan and poly-L-lysine in deionized water to obtain a mixed solution, adjust the pH value of the mixed solution to neutral, then add transglutaminase to the mixed solution to obtain Solution A. The mass percentage content of carboxymethyl chitosan in the mixed solution is 8.5% to 12%, and the mass percentage content of poly-L-lysine is 6.7% to 9.5%. Adjust the amount of transglutaminase based on the mass of recombinant collagen in Step two, with no less than 0.08 g of transglutaminase per gram of recombinant collagen;

Step two: Dissolve recombinant collagen and tetra-arm PEG-aldehyde in deionized water to obtain Solution B. The content of recombinant collagen in Solution B is adjusted according to the required time for wound healing, with a mass percentage content of recombinant collagen not less than 8%, preferably 8% to 15%, and the mass percentage content of tetra-arm PEG-aldehyde is 8% to 12%.

Step 3: Utilize a three-way valve and syringe to mix the A solution from Step 1 and the B solution from Step 2 in a volume ratio of 1:1 to obtain a recombinant collagen hydrogel tissue adhesive for sutureless wound healing. The Schiff base reversible bond formed by amino groups and aldehyde groups forms within ten seconds, constituting the first network of the hydrogel tissue adhesive; subsequently, amidation bonds are slowly formed between glutamine and amino groups under the action of transglutaminase, forming the second network of the hydrogel tissue adhesive.

### Example 1

The preparation method of the recombinant collagen hydrogel tissue adhesive for sutureless wound healing in this example specifically includes the following steps:

Step 1: At room temperature, dissolve 0.10 g of carboxymethyl chitosan and 0.08 g of polylysine in 1 mL of deionized water, stir and mix at 400 rpm for 2 hours until dissolved, obtaining a mixed solution, adjust the pH value of the mixed solution to neutral, then add 0.008 g of TG enzyme (transglutaminase) to the mixed solution, and mix to obtain the A solution;

Step 2: Dissolve 0.1 g of recombinant collagen and 0.10 g of tetra-arm PEG-aldehyde in 1 mL of deionized water, adjust the pH to neutral, to obtain the B solution;

Step 3: Utilize a three-way valve and syringe to mix the A solution from Step 1 and the B solution from Step 2 in a volume ratio of 1:1 to obtain a recombinant collagen hydrogel tissue adhesive for sutureless wound healing.

### Example 2

The preparation method of the recombinant collagen hydrogel tissue adhesive for sutureless wound healing in this example specifically includes the following steps:

Step 1: At room temperature, dissolve 0.12 g of carboxymethyl chitosan and 0.09 g of polylysine in 1 mL of deionized water, stir and mix at 300 rpm for 2 hours until dissolved, obtaining a mixed solution, adjust the pH value of the mixed solution to neutral, then add 0.008 g of TG enzyme to the mixed solution, and mix to obtain the A solution;

Step 2: Dissolve 0.1 g of recombinant collagen and 0.12 g of tetra-arm PEG-aldehyde in 1 mL of deionized water, adjust the pH to neutral, to obtain the B solution;

Step 3: Utilize a three-way valve and syringe to mix the A solution from Step 1 and the B solution from Step 2 in a volume ratio of 1:1 to obtain a recombinant collagen hydrogel tissue adhesive for sutureless wound healing.

### Example 3

The preparation method of the recombinant collagen hydrogel tissue adhesive for sutureless wound healing in this example specifically includes the following steps:

Step one: At room temperature, dissolve 0.15 g of carboxymethyl chitosan and 0.10 g of poly-L-lysine in 1 mL of deionized water, stir and mix at 200 rpm for 3 hours until dissolved, obtain a mixed solution, adjust the pH value of the mixed solution to neutral, then add 0.01 g of transglutaminase to the mixed solution, and mix to obtain Solution A;

Step two: Dissolve 0.1 g of recombinant collagen and 0.12 g of tetra-arm PEG-aldehyde in 1 mL of deionized water, adjust the pH to neutral, to obtain Solution B;

Step three: Using a three-way valve and syringe, mix Solution A from Step one and Solution B from Step two uniformly in a volume ratio of 1:1 to obtain the recombinant collagen hydrogel tissue adhesive for sutureless wound healing.

### Example 4

The preparation method of the recombinant collagen hydrogel tissue adhesive for sutureless wound healing in this example specifically includes the following steps:

Step one: At room temperature, dissolve 0.12 g of carboxymethyl chitosan and 0.10 g of poly-L-lysine in 1 mL of deionized water, stir and mix at 200 rpm for 3 hours until dissolved, obtain a mixed solution, adjust the pH value of the mixed solution to neutral, then add 0.008 g of transglutaminase to the mixed solution, and mix to obtain Solution A;

Step two: Dissolve 0.1 g of recombinant collagen and 0.15 g of tetra-arm PEG-aldehyde in 1 mL of deionized water, adjust the pH to neutral, to obtain Solution B;

Step three: Using a three-way valve and syringe, mix Solution A from Step one and Solution B from Step two uniformly in a volume ratio of 1:1 to obtain the recombinant collagen hydrogel tissue adhesive for sutureless wound healing.

### Example 5

The preparation method of the recombinant collagen hydrogel tissue adhesive for sutureless wound healing in this example specifically includes the following steps:

Step one: At room temperature, dissolve 0.15 g of carboxymethyl chitosan and 0.12 g of poly-L-lysine in 1 mL of deionized water, stir and mix at 200 rpm for 3 hours until dissolved, obtain a mixed solution, adjust the pH value of the mixed solution to neutral, then add 0.016 g of transglutaminase to the mixed solution, and mix to obtain Solution A;

Step two: Dissolve 0.2 g of recombinant collagen and 0.15 g of tetra-arm PEG-aldehyde in 1 mL of deionized water, adjust the pH to neutral, to obtain Solution B;

Step three: Using a three-way valve and syringe, mix Solution A from Step one and Solution B from Step two uniformly in a volume ratio of 1:1 to obtain the recombinant collagen hydrogel tissue adhesive for sutureless wound healing.

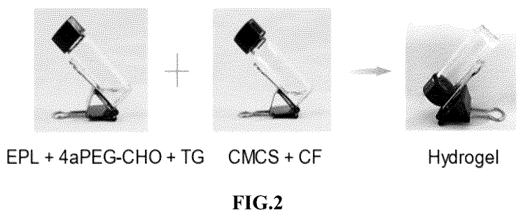

Performance evaluation: After adding solutions A and B to small vials, mix them evenly and invert to observe the formation of hydrogel. The results are shown in FIG. 2, indicating that the two liquids form a gel state at room temperature after mixing.

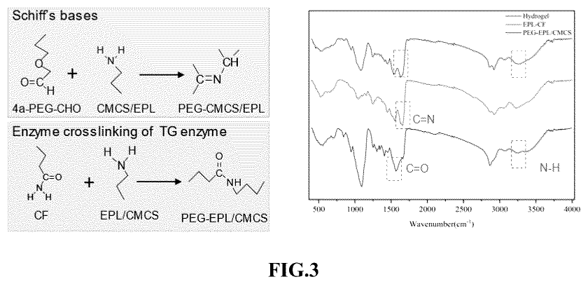

Infrared absorption spectra of freeze-dried hydrogel tissue adhesives were measured using a Fourier Transform Infrared Spectrometer (FT-IR). Test conditions included a resolution of 4 cm⁻¹, 32 scans, and a wavenumber range of 400-4000 cm⁻¹. The results are shown in FIG. 3, revealing weak infrared absorption for PEG-EPL/CMCS between 1690-1640 cm\hat{ }-1 (C═N), while EPL-CF shows a C═O bond absorption peak at 1670 cm⁻¹ and an N—H (amide bond) absorption peak at 3430 cm⁻¹. The FT-IR spectrum of PEG-EPL/CMCS-CF displays characteristic peaks at 1660-1690 cm⁻¹ and 3240 cm⁻¹ corresponding to amide and imine, indicating the formation of both networks.

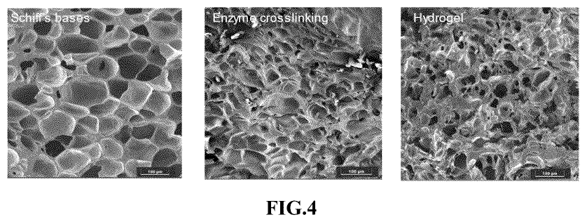

Samples of freeze-dried hydrogel tissue adhesives, approximately 2 mm thick, were cut and attached to the sample stage with conductive adhesive. Subsequently, the hydrogels were sputter-coated with gold and examined under a scanning electron microscope (SEM) to observe their microstructure. The results are shown in FIG. 4, demonstrating a porous three-dimensional network structure for the hydrogel tissue adhesive, with smaller pores compared to Schiff base single-network hydrogels.

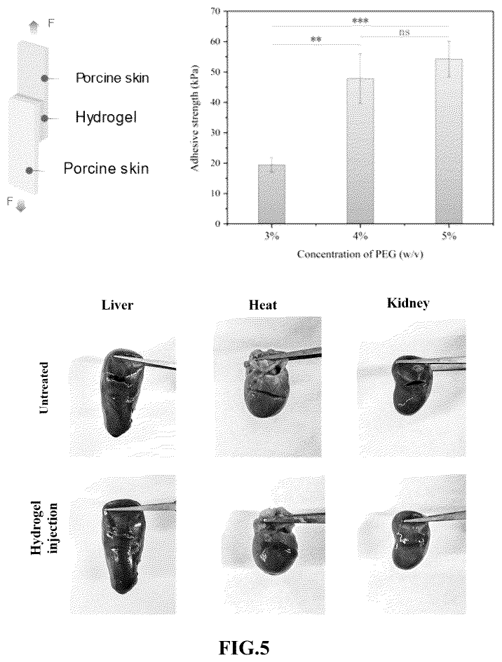

The adhesive properties of hydrogel tissue adhesives prepared in Examples 1, 4, and 5 of this invention were tested using a universal material compression machine. Prior to each test, 1 mL of hydrogel tissue adhesive was injected onto pig skin (50 mm long, 5 mm wide, 2 mm thick), and a lap shear test was conducted at a testing speed of 1 mm/min. Additionally, the compressive strength of the hydrogels was analyzed. Hydrogels were prepared in cylindrical molds (diameter 10 mm, height 5 mm) and compressed at a rate of 1 mm/min. Each test was repeated at least three times. For cyclic compression tests, cylindrical hydrogels (diameter 10 mm, height 5 mm) were cyclically compressed to 50% strain and returned to their initial height. The results are shown in FIG. 5, showing that as the proportion of aldehyde groups in the hydrogel system increases, the adhesive strength to pig skin also increases, reaching a maximum of 58.7 kPa. Furthermore, the hydrogels can tightly adhere to various tissues, including liver, heart, and kidney, achieving secure wound closure.

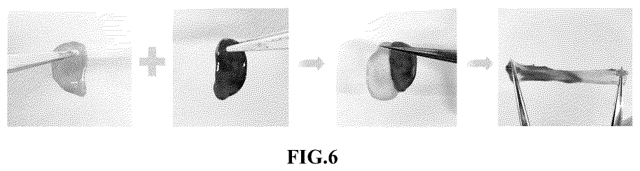

Using the method of this invention, two differently colored hydrogel tissue adhesives were prepared using dyes. The hydrogels were cut along a symmetrical line with a sterile surgical knife, and the two differently colored hydrogels were aligned along the incision. After some time, the self-healing property was observed by stretching. The results are shown in FIG. 6, indicating that due to the reversibility of the Schiff base bonds formed between aldehyde and amino groups, the hydrogels maintain their integrity under gravity and can be stretched like intact hydrogels.

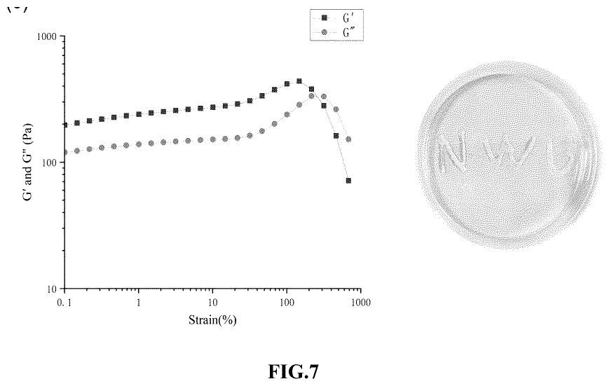

Use a rotational rheometer to evaluate the rheological properties of hydrogels at room temperature. Place the prepared hydrogel tissue adhesive samples on the plate of the rotational rheometer. Perform a strain-sweep rheological test on PEG-EPL-CF hydrogels to assess injectability. Additionally, conduct step-strain rheological tests between 5% and 500% strain to evaluate the self-healing properties of the hydrogels at the micro-level. Conduct a strain sweep on the pre-gel solution to verify linear response. The gel point is determined as the time when the storage modulus (G′) exceeds the loss modulus (G″). Oscillatory rheometry measurements are performed under linear viscoelastic conditions at 37° C. Results are shown in FIG. 7, indicating that when a 318% strain is applied to the hydrogel tissue adhesive, its internal network collapses, exhibiting a viscous liquid state, demonstrating the injectability of the hydrogel tissue adhesive.

To determine the in vitro cytocompatibility of the hydrogel tissue adhesive, co-culture the hydrogel with cell culture medium for 12 hours, then add 10\hat{ }5 cells/mL L929 cell suspension (100 μL) to a 96-well plate. After 12 hours, remove the medium and add 100 μL of hydrogel extract for incubation. At 24, 48, and 72 hours, add 10 μL of CCK-8 solution. After incubating in the dark at 37° C. for 2 hours, record the absorbance values at 450 nm using a microplate reader.

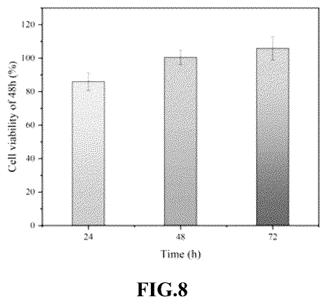

OD sample: culture medium containing cells, CCK-8, toxic substances; OD control: culture medium containing cells, CCK-8, no toxic substances; OD blank: culture medium without cells and toxic substances, CCK-8. Results are shown in FIG. 8, indicating that the cytotoxicity of the hydrogel tissue adhesive is below 80%, showing good biocompatibility.

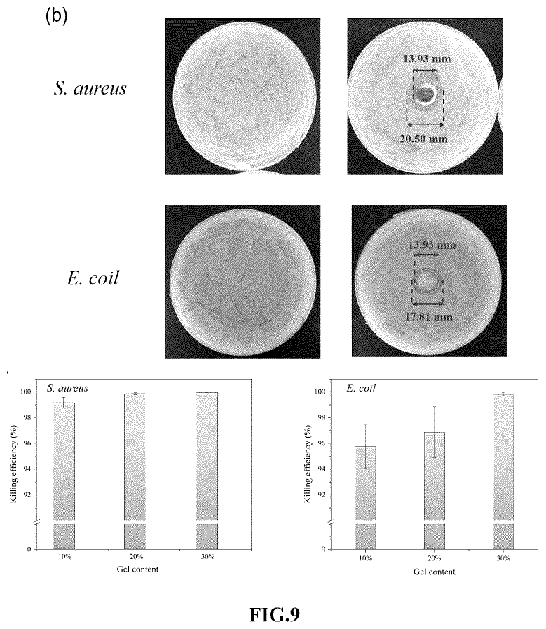

To evaluate the antimicrobial performance of the hydrogel tissue adhesive, apply 100 μL of bacterial suspension (10\hat{ }6 CFU/mL), and perform an inhibition zone assay. Place the hydrogel tissue adhesive at the bottom of a test tube, and add bacterial suspensions (10\hat{ }6 CFU/mL) at different volume percentages (10%, 20%, 30%) onto the hydrogel tissue adhesive. Incubate the test tubes at 37° C. for 12 hours. After imaging, transfer the supernatant to a 96-well plate and measure the OD values at 600 nm using a microplate reader (with samples without hydrogel as controls). Simultaneously, spread the post-co-culture bacterial suspension, count colonies after 12 hours to calculate the bactericidal rate. Results are shown in FIG. 9, indicating that the hydrogel tissue adhesive has excellent antimicrobial efficacy, with MBCs (bactericidal rate >99.9%) for Staphylococcus aureus and Escherichia coli being 20% (v/v) and 30% (v/v), respectively.

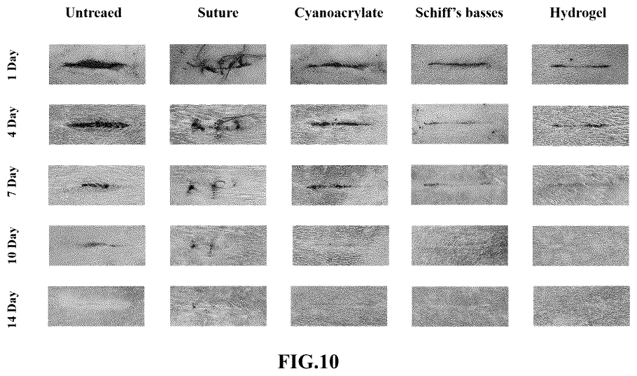

To further investigate the potential of hydrogels for wound closure, an SD rat full-thickness skin incision model (male, incision length=2 cm) was used. Mix solutions A and B uniformly and inject them onto the incision, then pinch with fingers for 2 minutes. Compare with surgical sutures, cyanoacrylate glue, and Schiff base single-network hydrogel. Results are shown in FIG. 10, indicating that the hydrogel tissue adhesive has good wound adhesion effects. The double-network hydrogel group promotes collagen deposition at the wound site and heals faster than the Schiff base single-network hydrogel group. In contrast, the suture group heals more slowly and is prone to inflammation, while the cyanoacrylate glue is brittle, making the incision prone to secondary cracking.

The above description pertains only to exemplary embodiments of the present invention and does not impose any limitations on it. Any simple modifications, variations, or equivalent structural changes made based on the technical essence of the invention to the aforementioned embodiments are still within the scope of protection of the present invention's technical solution.

What is claimed is:

1. A method for preparing a recombinant collagen hydrogel tissue adhesive for sutureless wound healing, comprising the following steps:
S1. At room temperature, carboxymethyl chitosan and poly-L-lysine are dissolved in deionized water to obtain a mixed solution, the pH of the mixed solution is adjusted to neutral, and transglutaminase is added to the mixed solution to obtain Solution A;
S2. Recombinant collagen and tetra-arm polyethylene glycol aldehyde are dissolved in deionized water to obtain Solution B;
S3. Solution A from Step one is mixed with Solution B from Step two to obtain a recombinant collagen hydrogel tissue adhesive for sutureless wound healing.

2. The method according to claim 1, wherein the mass percentage content of carboxymethyl chitosan in the mixed solution of Step one is 8.4% to 12%, and the mass percentage content of poly-L-lysine is 6.7% to 9.5%.

3. The method according to claim 1, wherein the amount of transglutaminase used in Step one is adjusted according to the mass of recombinant collagen, with no less than 0.08 g of transglutaminase per gram of recombinant collagen.

4. The method according to claim 1, wherein the content of recombinant collagen in Solution B of Step two is no less than 8%, and the mass percentage content of tetra-arm polyethylene glycol aldehyde being 8% to 12%.

5. The method according to claim 4, wherein the mass percentage content of recombinant collagen in Solution B is 8% to 15%.

6. The method according to claim 1, wherein the recombinant collagen in Step two is CF-1552 type I recombinant collagen, and the molecular weight of tetra-arm polyethylene glycol aldehyde is 10000.

7. The method according to claim 1, wherein the volume ratio of Solution A to Solution B in Step three is 1:1.

8. The method according to claim 1, wherein, in Step three, a three-way valve and a syringe are used to mix Solution A and Solution B evenly.
